(19) United States
(12) Patent Application Publication
(10) Pub. No.: US 2008/0194995 A1
(43) Pub. Date: Aug. 14, 2008

(54) Method For Analyzing Support Pressure

(76) Inventor: Johanna Hendrika Grady-Van Den Nieuwboer, Haaksbergen (NL)

Correspondence Address: LEVINE BAGADE HAN LLP, 2483 EAST BAYSHORE ROAD, SUITE 100, PALO ALTO, CA, 94303, US

(21) Appl. No.: 11/569,348

(22) PCT Filed: May 19, 2004
(86) PCT No.: PCT/NL2004/000361

§ 371 Date: Nov. 6, 2007

(51) Int. Cl. A61B 5/00

(52) U.S. Cl. 600/587

(57) ABSTRACT

The invention relates to a method for analyzing support pressure of a support for a human body, which method comprises the steps of: -providing an array of pressure sensors at the support for measuring pressure values; -registering pressure values measured by the pressure sensors during a time interval; -dividing the registered pressure values of at least one pressure sensor into at least two pressure ranges; and -calculating the contribution of each range over the time interval.

This invention relates to a method for analyzing support pressure of a support for a human body. In particular, with disabled people, who are bound to, for example, a wheel chair or a bed, specific physical problems can arise which are related to the way the body is supported.

The most occurring pressure related body complaint is pain.

Another severe problem is the development of pressure sores, which are the result of prolonged high local pressures. Also lack of stability of the human body in sitting and in lying down can result in problems. If the person being supported by a support surface, has to adapt his or her body position constantly or cannot correct a change in body position, this could have negative effects on the body resulting in tiredness, pressure sores (local overloading) and pain.

Supports, such as seats and back rests, are generally designed based on the expert knowledge of the designer. To provide a good support, it is sometimes difficult and might even need custom-made supports instead of standard supports in order to provide the necessary quality of support.

What is needed is a method for analyzing support pressure of a support for a human body, such that supports can be objectively and quickly evaluated, such that a user is quickly provided with the medically adequate support he or she needs.

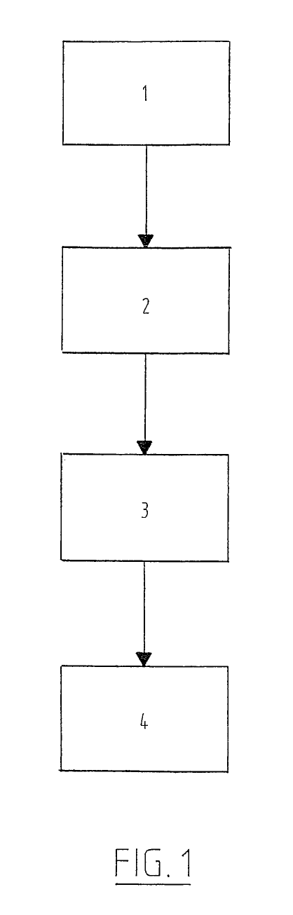

FIG. 1 is a flow chart of a method of the invention.

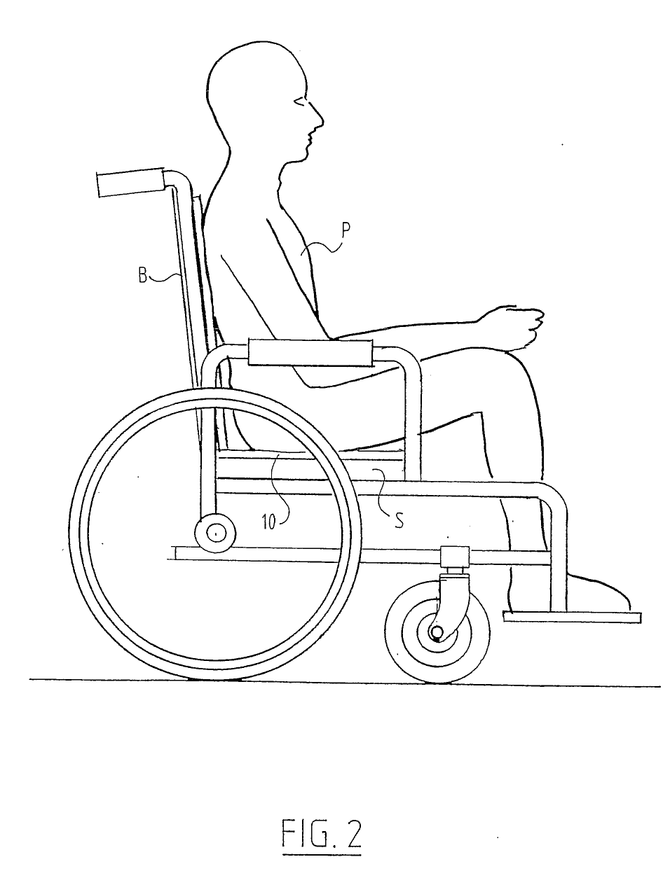

FIG. 2 is a side view of a person sitting on a support with a sensor mat therebetween.

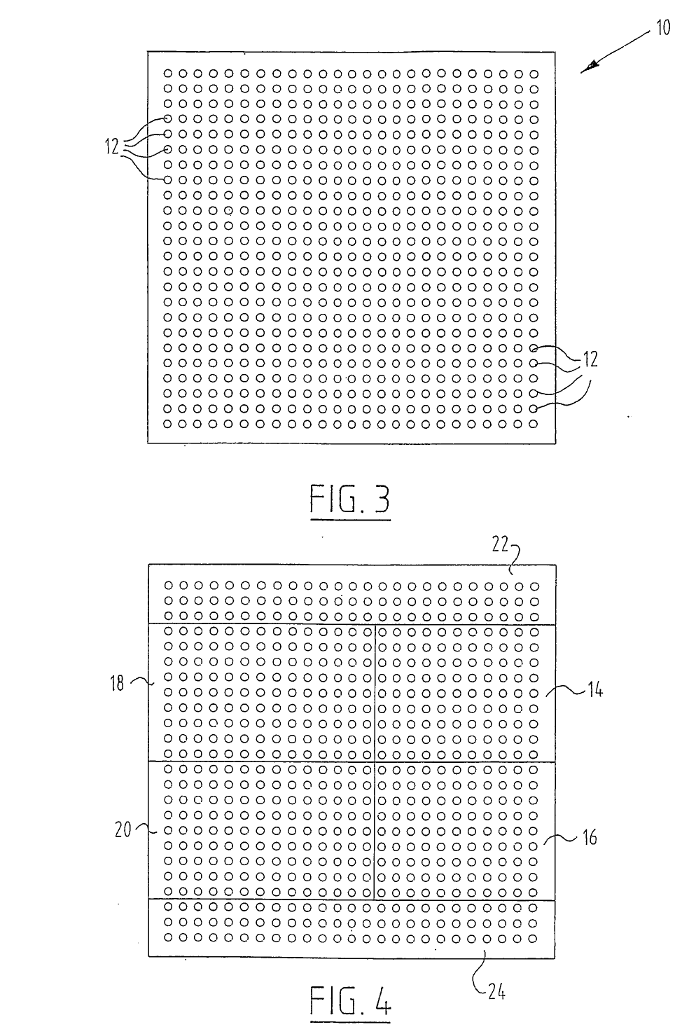

FIG. 3 is a top plan view of a sensor mat.

FIG. 4 is a top plan view of areas into which sensors can be divided.

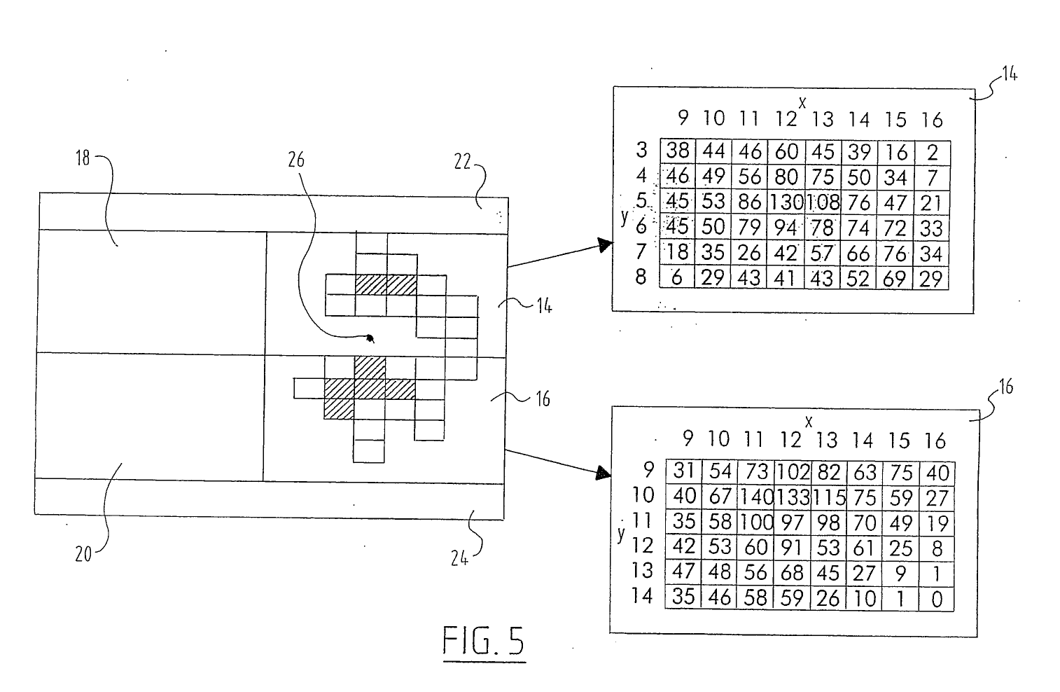

FIG. 5 is a graph representing pressure quantities per unit time.

This object is achieved by a method according to the invention, which comprises the steps of:

providing an array of pressure sensors at the support for measuring pressure values (step 1);

registering pressure values measured by the pressure sensors during a predefined time interval (step 2);

dividing the registered pressure values of at least one pressure sensor into at least two pressure ranges (step 3); and

calculating the contribution of each range over the predefined time interval (step 4).

Pressure sores are the result of high local pressures for a prolonged time. The human skin can cope with a sporadic high pressure peak, but it has to have sufficient time to recuperate. Therefore according to the invention the registered pressure values are divided into at least two pressure ranges, so that it can be determined whether a specific location of the support does or does not cause an unacceptable high pressure to the human body.

The pressure ranges are defined, so that it can be predicted when overload will result in complaints of pain, tiredness or instability and abnormalities, such as pressure sores. For example, diabetics already have a tendency to develop pressure sores, while people suffering from spasms can bear a higher load than an average person.

In a preferred embodiment, the calculated contribution of each range is expressed in an indicator. With this indicator, one can quickly determine which locations could provide problems and, if necessary, the support can be adjusted in order to remove these pressure points.

According to the most preferred embodiment of the invention, the registered pressure values are divided into three pressure ranges, wherein the first pressure range is defined as a pressure range below a certain pressure value threshold, in which no pressure related medical problems will arise; the second pressure range is defined as a range of certain pressure values in which pressure related medical problems may or may not arise, depending on the differential pathology; the third range is defined as pressure values above a certain pressure threshold in which pressure related medical problems will arise.

In a very preferred embodiment, the indicator is a combination of the calculated contribution of each range in terms of percentage. This provides for a standardized tool to evaluate a support.

In another embodiment of the method according to the invention, the registered values are divided into three pressure ranges, wherein a first pressure range is 0-60 mmHg, a second pressure range is 60-100 mmHg and a third pressure range is defined as pressures above 100 mmHg.

These three pressure ranges provide for a good differentiation between pressures, that could provide problems and pressures that do not form any risk.

In yet another embodiment of the invention, the method comprises the steps of:

dividing the support surface into at least two areas; and

combining the registered pressure values of sensors within an area.

Pressure measurements are carried out with a large number of sensors. In a conventional method, it is difficult to determine where the problem areas exist. By dividing the support surface into at least two areas and then combining the registered pressure values of sensors within those areas, one can quickly see if, within an area, a problem occurs and then one can concentrate on the specific sensors within that area to determine the exact location of the problem area.

Preferably, the dividing of the registered pressure values and the calculating of the contribution is based on the combined pressure values per area.

In another embodiment, the method comprises the steps of determining the sensors having registered measurements exceeding a predetermined value of a predetermined quantity. With this step, the critical locations are determined, so that one can concentrate on these areas.

Again, it is preferred to first divide the support surface into at least two areas and then divide the critical areas.

In yet another embodiment of the method according to the invention, the method comprises the steps of calculating the center of pressure based on the registered pressure values and the geometry of the array of pressure sensors. In order to provide a good support, the center of pressure should not shift much in time. By calculating the location of the x- and y-coordinates, this can be determined.

In still another embodiment, the method according to the invention comprises the steps of calculating the contact area based on the number of sensors measuring a pressure and the geometry of the array of pressure sensors. If the available body surface of the specific person is known, one can determine how much of this available body surface is actually supported. It also provides for a more effective and comfortable support.

Now, an example of an embodiment of the method according to the invention is elucidated.

For determining whether the seating system of a wheelchair for a disabled person provides sufficient support and does not cause any risk on pressure sores, a mat (10) comprising a large number of pressure sensors (12) is placed between the person (P) and the seat (S) and/or backrest (B) of the wheelchair, as shown in FIGS. 2 and 3. First of all, the person is instructed in accordance with a measurement protocol, which dictates which postures are allowable during the measurements and in between the measurements, and which are not. These instructions are necessary as a wrong posture or sudden movements can invalidate the measurements.

Then, during three intervals, measured pressures are collected during those three periods.

Of each sensor during each interval, a number of quantities are determined. Such quantities can be, for example, the maximum and minimum pressure, the average pressure and so on.

In order to present this information in a more simple way, the sensors are divided into a number of areas, which correspond to different areas of the human body. In this case, the areas are divided into two areas (14, 16) corresponding to the left and right buttock, two areas (18, 20) corresponding to the left and right legs, as shown in FIG. 4, and two areas (22, 24) relating to the left and right hip region. For each area (14, 16, 18, 20, 22, 24), it is determined whether such an area comprises a pressure load that exceeds a critical level. In such a case, it is indicated that an area is a problem area. This provides the operator an indication to examine the area in more detail by looking at the pressure load per sensor. In this way, the operator can see at a glance which area of the seat provides a problem.

In FIG. 5 the measured pressures for the two buttock areas (14, 16) are shown for each sensor. The x-coordinate and y-coordinate of each sensor is also shown. With this geometry of a pressure sensor mat, the x-coordinates and the measured pressures it is possible to determine the center of pressure (26).

When determining the quantities per sensor or per area, a specific quantity according to the invention is determined. For this quantity, the registered pressure values for a sensor or for an area are divided into three ranges. The first pressure range may, for example, be defined as 0-60 mmHg and provides no danger for any problem. The second pressure range may, for example, be defined as 60-100 mmHg and is a moderate risk for a problem. If the pressures are too long within this range, problems might occur. The third pressure range may, for example, be defined as above 100 mmHg and presents a high risk score. Such pressures almost instantly provide problems.

As already mentioned, the defined pressure ranges depend on the specific person. For this, a standard is defined in the art based on a medical validation.

After the division into these three pressure ranges, the contribution of each range over a measurement time interval is calculated. If, for example, the pressure is only for a very short time within the second range and for the rest of the time in the first range, this still does not provide any problem for the body of that specific person. However, if the pressures vary mainly within the second range, this would probably cause physical problems for the user of the support.

In order to present these calculated contributions in an indicator, the contributions are expressed in terms of a percentage. Then, the indicator is composed out of three digits, wherein each digit represents the percentage of the contribution of a respective area. For example, an indicator such as “172” indicates that the pressure varied for 10% of the measurement time interval within the first range, for 70% within the second range and for 20% within the third range.

By providing this indicator for each sensor, an operator can quickly see what is happening at a specific sensor location.

This is advantageous over the known state of the art in which only quantities, such as the maximum pressure value, the minimum pressure value and the average pressure value, are determined per measurement interval. If a pressure sensor registers a pressure peak for just a very short time, the maximum value will correspond to this pressure peak. However, if the rest of the time, the pressures are within the first range, this area will not provide a physical problem. In a conventional method, the measurements could provide a false reading. With this new indicator according to the invention, the operator can directly see how long a specific pressure was within one of the three ranges. This enables the operator to quickly determine problem areas and adjust the seating if necessary.

1. A method for analyzing support pressure of a support for a human body, which method comprises the steps of:
providing an array of pressure sensors at the support for measuring pressure values;
registering pressure values measured by the pressure sensors during a predefined time interval;
dividing the registered pressure values of at least one pressure sensor into at least two pressure ranges; and
calculating a contribution of each range over the predefined time interval.

2. The method according to claim 1, wherein the calculated contribution of each range is expressed in an indicator.

3. The method according to claim 2, wherein the indicator is a combination of the calculated contribution of each range in terms of a percentage.

4. The method according to claim 1, wherein the registered pressure values are divided into three pressure ranges, wherein a first pressure range is 0-60 mmHg, a second pressure range is 60-100 mmHg and a third pressure range is defined as pressures above 100 mmHg.

5. The method according to claim 1, comprising the steps of:
dividing the support surface into at least two areas; and
combining the registered pressure values of sensors within an area.

6. The method according to claim 5, wherein the dividing of the registered pressure values and the calculating of the contribution is based on the combined pressure values per area.

7. The method according to claim 1, comprising the step of determining sensors having registered pressure values exceeding a predetermined value of a predetermined quantity.

8. The method according to claim 7, comprising the steps of dividing the support surface into at least two areas; and determining areas having sensors with registered pressure values exceeding a predetermined value of a predetermined quantity.

9. The method according to claim 1, comprising the step of calculating the center of pressure based on registered x- and y-coordinates of the pressure values and the geometry of the array of pressure sensors.

10. The method according to claim 1, comprising the step of calculating the contact area based on the sensors measuring a pressure and the geometry of the array of pressure sensors.

11. A method for analyzing support pressure of a support for a human body, which method comprises the steps of:
providing at least one pressure sensor for measuring pressure values;
registering pressure values measured by the pressure sensor during a time interval;
dividing the registered pressure values of the pressure sensor into at least two pressure ranges; and
calculating a contribution of each range over the time interval.

12. The method according to claim 11, where the pressure sensor is one of an array of pressure sensors and the registering pressure values are measured by the array of pressure sensors during the time interval.
